(19) United States
(12) Patent Application Publication
(10) Pub. No.: US 2007/0060890 A1
(43) Pub. Date: Mar. 15, 2007

(54) Catheter safety tube needle retraction system

(75) Inventor: Michael J. Cuppy, Burnsville, MN (US)

Correspondence Address: FULBRIGHT & JAWORSKI L.L.P., 80 SOUTH EIGHTH STREET, SUITE 2100, MINNEAPOLIS, MN, 55402, US

(73) Assignee: Cuppy Medical Products, Inc.

(21) Appl. No.: 11/517,770

(22) Filed: Sep. 8, 2006

Related U.S. Application Data
(60) Provisional application No. 60/715,564, filed on Sep. 8, 2005.

(51) Int. Cl. A61M 5/178

(52) U.S. Cl. 604/164.01; 600/576; 604/164.08

(57) ABSTRACT

A tube-based catheter and needle system providing spring retraction of a needle subsequent to patient access. A retraction spring is connected to the needle and maintained in an energized state prior to activation. Rotation of a tube portion relative to the needle causes a spring release and retraction of the needle into a safety tube.

### RELATED APPLICATIONS

This application claims the benefit of U.S. Provisional Application Ser. No. 60/715,564, filed Sep. 8, 2005.

### TECHNICAL FIELD

The invention relates to an intravenous access system and more particularly to a catheter and safety tube needle retraction system.

### BACKGROUND OF THE INVENTION

Catheters are commonly used to provide intravenous access, for example to obtain a blood sample from a patient. Catheters are typically utilized with a needle, wherein the needle is used to puncture the skin and position the catheter within a vein. The needle is then retracted and the catheter remains in place. Needle safety tubes have been used in conjunction with catheters to protect the user from accidental needle sticks during needle retraction and disposal. Prior to coupling a catheter to another device or line, the user often first tamponade the vein with gauze or other material to prevent excessive bleeding and then rapidly retract the needle and guide it into a safety tube without incurring a needlestick or blood born exposure. It is during this needed retraction and tamponading that a number of needlesticks and blood born exposures occur.

Some safety catheters use a trigger that actuates a spring to retract the needle into a safety tube. Such retraction systems have significant limitations, including inadvertent exposure to blood as the user may be exposed to blood spray or blood “back-splash” during a spring retraction process. Unsuccessful tamponading of the vein may also lead to blood exposures during IV line attachment. Another limitation is that the trigger is often not adequately protected from inadvertent actuation. Inadvertent actuation results in needle retraction before the catheter is successfully inserted, causing significant blood-born exposures and unsuccessful IV attempts.

Consequently, there is a need for a safety catheter needle retraction system that protects users from both needlesticks and blood-born exposures, and also improves the simplicity and dependability of the needle retraction process for users.

### BRIEF SUMMARY OF THE INVENTION

The present invention is directed to a safety catheter needle retraction system having a selectively retractable needle. An embodiment of the present invention includes a trigger assembly requiring the user to deliberately rotate a portion of the device in order to actuate the mechanism and retract the needle. The use of a safety stop prevents a user from separating the catheter from the safety tube without first retracting the needle. Blood back-splash exposures and spring strength are minimized as the needle retracts through a self-sealing injection port, which “wicks” or “squeegees” the blood off the needle as it passes through the membrane.

A retraction mechanism of an embodiment of the safety catheter needle retraction is spring actuated. The retraction spring provides needle extraction and containment within the safety tube. The safety tube assembly allows the user to easily grasp and manipulate the catheter during an IV start, and permits easy storage in drug boxes and crash carts.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1 is a side elevation view of the safety catheter system according to the present invention;

FIG. 2 is a perspective illustration of a piercing needle and needle carrier of the catheter system of FIG. 1;

FIG. 3 is a perspective illustration of components of a retraction spring assembly;

FIG. 4 is a cross-section view of a safety tube assembly and trigger pins of the catheter system in FIG. 1;

FIG. 5 is a cross section view of the safety tube assembly, piercing needle and retraction spring of the catheter system in FIG. 1;

FIG. 6 is a front elevational view of the safety tube assembly of FIG. 5;z

FIG. 7 is a perspective illustration of the safety tube assembly of FIG. 5;

FIG. 8a-FIG. 8e depict operation of the catheter and safety tube assembly;

FIG. 9a-FIG. 9d depict operation of the safety tube trigger and needle retraction into safety tube;

FIG. 10 is a cross-section view of the safety tube assembly and trigger of the catheter system of FIG. 1;

FIG. 11 is a perspective illustration of portions of the safety tube assembly and needle-tip cover of FIG. 4; and

FIG. 12 is a perspective illustration of portions of the safety tube assembly, needle and needle retraction system according to one embodiment of the present invention.

### DETAILED DESCRIPTION OF THE INVENTION

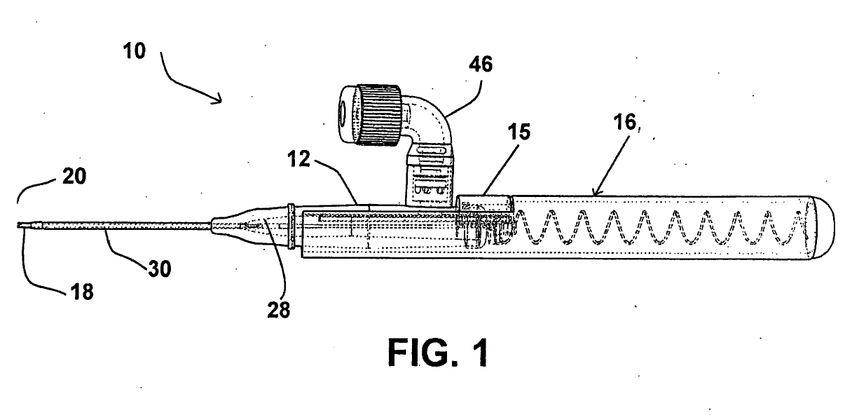

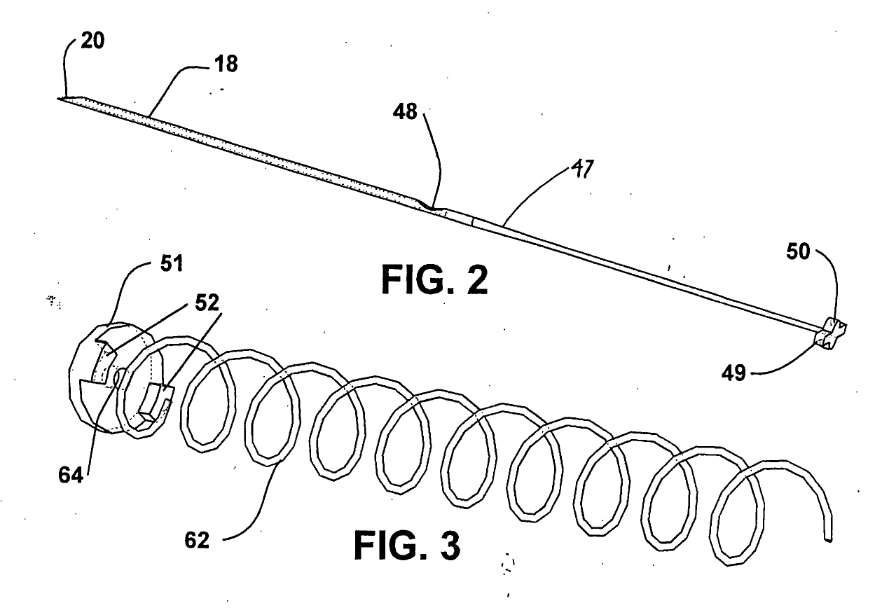

An embodiment of a catheter system according to the present invention is shown in FIGS. 1-12 and is referenced herein by the numeral 10. Referring to FIG. 1, catheter system 10 includes an over-the-needle type “closed hub” catheter assembly 12 with a connecting U-port coupling 46 disposed at the front or proximal end of the catheter system 10. Safety tube assembly 16 is disposed at the rear or distal end of catheter system 10. Catheter assembly 12 includes a relatively long venous-gauge coring-tipped needle 18 having an angled distal point 20 for percutaneous penetration of a patient's skin and vascular system. As described in more detail hereinafter, safety tube assembly 16 includes a slide-biased needle-tip cover 15 providing needle protection. Additional aspects of catheter assembly 12 and U-port coupling 46 are disclosed in U.S. Ser. No. 10/797,526, entitled “Vascular Access Device and Method of Using Same,” the entire disclosure being incorporated by reference herein.

Referring to FIGS. 1-3, needle 18 defines side aperture 48 positioned near the proximal end of catheter cannula 30 such that the side aperture 48 vents blood into blood flash chamber 28 in a manner so as to be visible through a transparent distal region of flash chamber 28. Side aperture 48 is shown for illustrative purposes as an elongated opening or groove in needle 18, but it may be appreciated that a small bore hole oriented, for example, perpendicular to or at an angle relative to the longitudinal axis of needle 18 may be sufficient in other applications. A portion 47 of needle 18 is tapered in order to reduce needle diameter. An end of needle 18 is fixedly attached to and partially embedded into needle carrier 50 which is sized and geometrically configured so as to be engaged and slidably received within safety tube assembly 16 as described in further detail below, and to maintain a proper orientation of the needle 18 and distal point 20 within housing 28 and catheter tube 30. Needle carrier 50 further defines arms 49 extending outwardly in opposing directions from the center of needle carrier 50. Arms 49 operate in conjunction with needle carrier arm stops 52, shown in FIG. 3, extending radially inward from retraction spring base 51. Needle carrier arm stops 52 act to stabilize and prevent any rotation of piercing needle 18 attached to needle carrier 50, prior to needle retraction. Retraction spring base 51 is sized and geometrically configured so as to be engaged and slidably received within safety tube assembly 16. Retraction spring base 51 receives a portion of piercing needle 18 through aperture 64 and needle carrier 50 and needle carrier arms 49 seats between needle carrier arm stops 52. Retraction spring 62 is attached and embedded in one of the needle carrier arm stops 52.

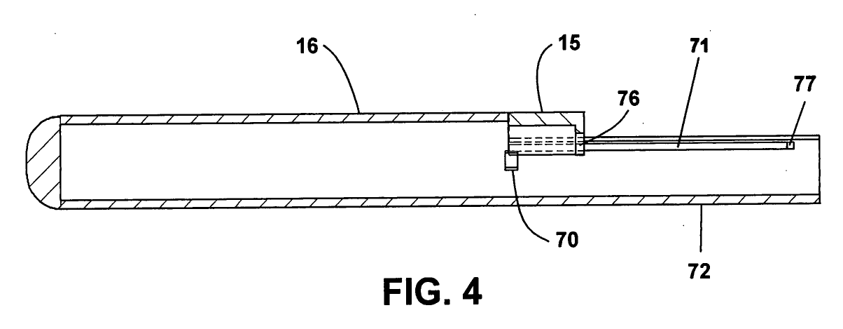

Referring particularly to FIG. 4, additional components of safety tube 16 are shown in greater detail. In one embodiment, two trigger pin projections 70 are situated within safety tube 16 such that needle carrier arms 49 rest against the trigger pins 70 and are used in restraining and then releasing needle carrier 50 into safety tube 16. A lubricant or low friction surface(s) may be provided at the interface between arms 49 and pins 70 to facilitate a sliding connection therebetween. One end of safety tube 16 includes a catheter gripping area 72 adapted to provide a user with a non-slippery surface permitting a user to safely grasp catheter device 10 in order to insert the cannula 30 into a patient. Catheter-gripping surface 72 may have a roughened, textured and/or raised surface to aid the user in gripping device 10 during use. Catheter grip area 72 is preferably provided within a region of tube 16 which, when rotated during trigger actuation, will also act as a blood splash shield during a needle retraction procedure. Two needle-tip slide cover channels 71 are provided generally opposite to catheter grip area 72. Channels 71 carry needle-tip slide cover 15 which, in this embodiment, has hemispherical protrusions that slide within slide cover channels 71. Needle-tip slide cover 15 is pulled along needle-tip slide cover channels 71 by needle cover tab safety stop 76 which is engaged with a tab (not shown) on catheter assembly 12 when advanced into the vasculature of the patient. When the catheter is fully advanced, needle-tip slide cover 15 locks into needle-tip cover locking detent 77 located at the distal end of safety tube 16. In this manner, needle-tip slide cover 15 acts to protect piercing tip 20 of needle 18.

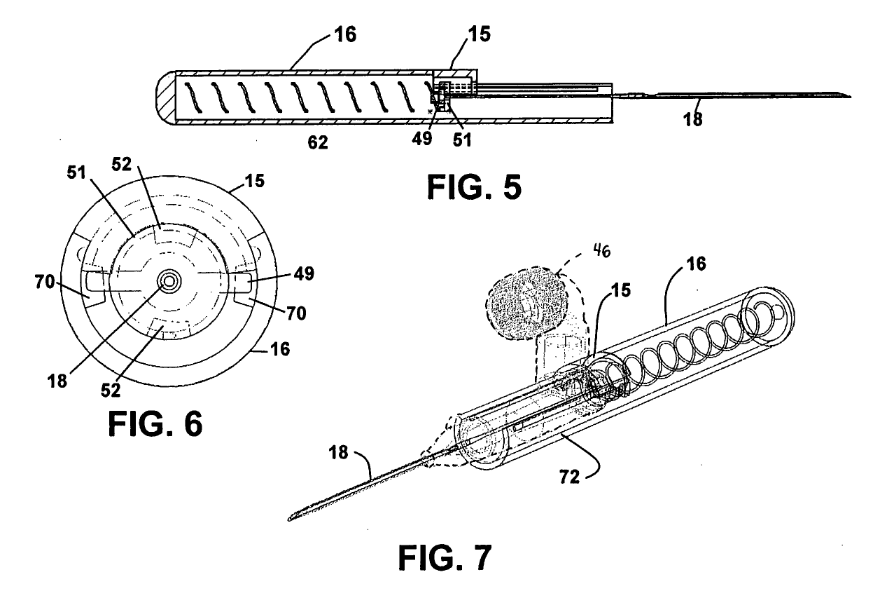

Referring particularly to FIGS. 5-7, in one embodiment, safety tube 16 can be seen in a “cocked” or pre-use state. To aid in clarity, closed hub catheter 46 is not shown mounted over the piercing needle 18 and nestled near the gripping surface area 72. FIG. 6 shows how needle carrier arms 49 rest against trigger pins 70 to restrain needle 18 until the safety tube 16 is rotated to free trigger pins 70 from the back of needle carrier arms 49.

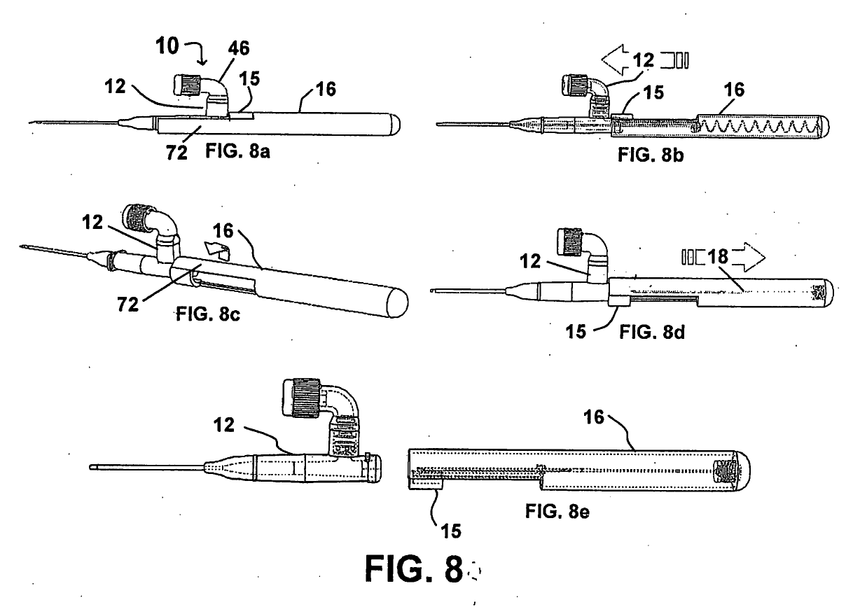

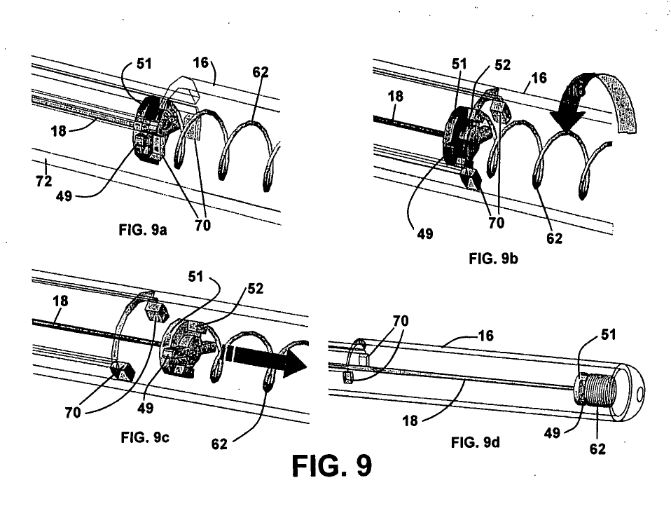

Referring particularly to FIGS. 8a-8e, one embodiment of catheter system 10 is shown composed of an over-the-needle type “closed hub” safety catheter assembly 12 with U-port coupling 46 disposed at the front or distal end of catheter system 10 and safety tube assembly 16 disposed at the rear or proximal end of the catheter system 10. FIGS. 8a thru 8e show the progression of steps required to insert catheter assembly 12 and then activate needle retraction system. FIG. 8a shows catheter system 10 as it is removed from a sterile packaging and with a needle guard removed (ready for first use). FIG. 8b depicts the catheter 12 advancing into vasculature while needle cover tab safety stop 76 is pulling needle-tip slide cover 15 along to the distal end of safety tube 16. FIG. 8c shows safety tube system 16 rotated 45 degrees. This rotation converts catheter gripping area 72 into a blood splash shield as the needle is retracted out of catheter 12. FIG. 8d shows the safety tube fully rotated 180 degrees. Ideally, at approximately 170 degrees, the safety tube rotation triggers the needle retraction system to retract needle 18 into safety tube 16. FIG. 8e shows catheter system 12 separated from safety tube 16 and piercing needle 18 safely stored within safety tube 16 with the needle-tip 20 protected by needle-tip cover 15.

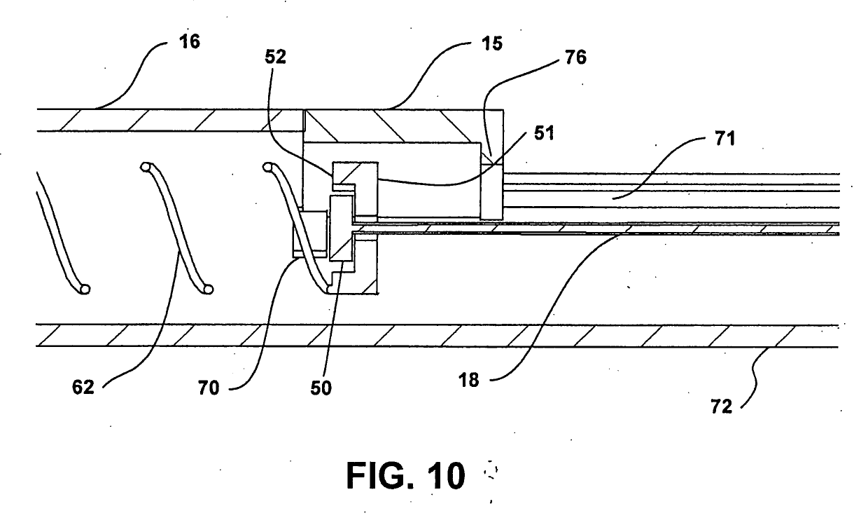

FIGS. 9a-9d illustrate aspects of operation of an embodiment of the present invention. In FIG. 9a, the triggering system is “cocked” and in its initial orientation. Needle 18 is restrained from retraction parts 51 and 62 by needle carrier arms 49 contacting trigger pins 70. FIG. 9b shows the initial rotation of needle safety tube 16. This rotation also rotates attached trigger pins 70, when trigger pins 70 rotate to clear contact with needle carrier arms 49, piercing needle 18 retracts. Needle carrier arm stops 52 act to restrain shadow rotation of needle carrier arms 49 with trigger pins 70, during rotation, needle carrier arms 49 rotate with trigger pins 70 until needle carrier arms 49 contact needle carrier arm stops 52, allowing arms 49 to stop rotation and clear the trigger pins 70. FIG. 9c illustrates needle carrier arms 49 now clear of trigger pins 70 and retraction spring base 51 and attached pre-stretched/flexed spring 62 starting to retract (arrow) piercing needle 18. FIG. 9d shows piercing needle 18 fully retracted into safety tube 16. Spring 62 is then fully compressed carrying retraction spring base 51, needle carrier 50 and piercing needle 18 to the proximal end of safety tube 16. The spring tension of retraction spring 62 will hold piercing needle 18 within the confines of safety tube 16.

FIG. 10 illustrates a partial cross-sectional view of components of the safety tube trigger mechanism of FIGS. 9a-9d. Pre-tensioned retraction spring 62 is attached to retraction spring base 51 by mounting into one of needle carrier arm stops 52. Needle-tip cover 15 is shown in its retracted position and engaging needle cover drag tab 76. As catheter 12 is advanced, a tab on the back of catheter 12 (not shown) engages needle cover drag tab 76 and pulls needle-tip cover 15 along needle-tip cover slide channels 71 and into its locked position at the distal end of safety tube 16. FIG. 10 shows that trigger pins 70 restrain needle 18 by contact with needle carrier 50.

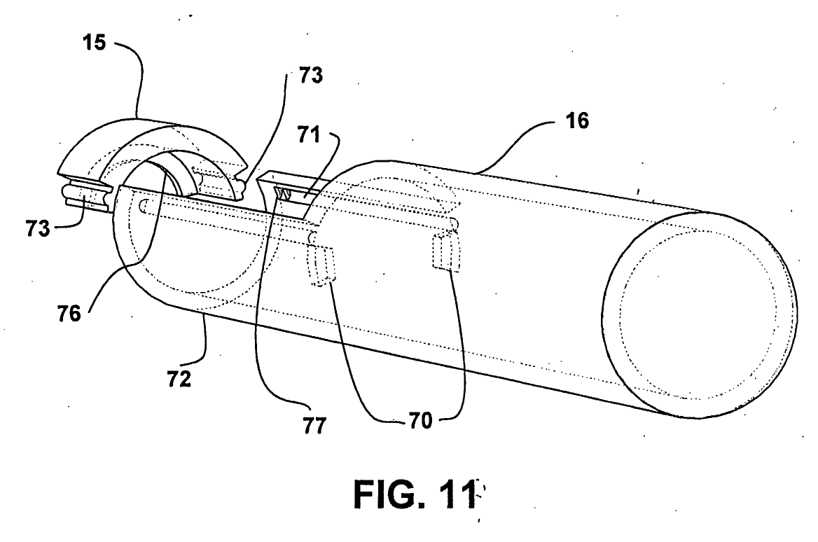

FIG. 11 discloses one embodiment of the needle-tip cover 15 and its relationship with safety tube 16. Two parallel needle-tip cover slide channels 71 are provided within an upper portion of gripping area 72. Slide channels 71 allows needle-tip cover 15 to travel from its initial position, shown in FIG. 10, at the proximal end of gripping area 72 to its locked position at the distal end of gripping area 72. Needle-tip cover 15 locks into the needle-tip cover lock/stop 77 to prevent the needle-tip cover from retracting and exposing the piercing needle-tip 20. Needle cover advancement projection 76 is shown near one end of needle-tip cover 15.

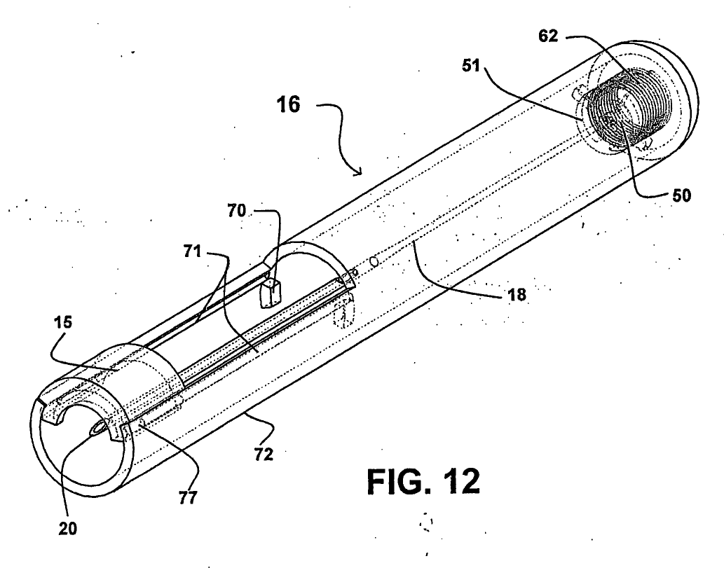

FIG. 12 discloses one embodiment of the retracted state of the components of safety tube assembly 16. This view shows needle-tip cover 15 in its locked state utilizing the needle-tip cover locks 77, and protecting piercing tip 20 of needle 18. Retraction spring 62 holds piercing needle 18 within the confines of safety tube 16. Safety tube 16 can then be safely disposed.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A catheter system comprising:
a catheter assembly having a needle for percutaneous access of a patient; and
a safety tube assembly for capturing and removal of said needle from said catheter assembly, said safety tube assembly comprising:
a tube;
a needle carrier connected to said needle, said carrier being slidably received within a portion of said tube; and
a retraction spring connected at one end to said tube, said retraction spring being maintained in a stretched state while said needle carrier engages a tube projection, and said spring retracting said needle into said tube when said tube is rotated relative to said needle carrier to remove said tube projection engagement.

2. The catheter assembly of claim 1 wherein said needle carrier includes at least one outwardly extending arm adapted to engage said tube projection.

3. The catheter assembly of claim 2 wherein said needle carrier includes a pair of generally opposite outwardly extending arms adapted to engage a pair of tube projections within a tube interior.

4. The catheter assembly of claim 3 wherein said needle carrier engages a retraction spring base.

5. The catheter assembly of claim 4 wherein said retraction spring base slidably receives a portion of said needle and said retraction spring is connected to said spring base.

6. The catheter assembly of claim 5 wherein said spring base includes a needle carrier arm stop adapted to engage said at least one outwardly extending arm of said needle carrier.

7. The catheter assembly of claim 1 further comprising a catheter-gripping area having a surface treatment to promote assembly manipulation.

8. The catheter assembly of claim 7 wherein said area includes a textured or raised surface or both.

9. The catheter assembly of claim 8 wherein said area is located at a distal end of said tube.

10. The catheter assembly of claim 1 further comprising a sliding cover.

11. The catheter assembly of claim 10 wherein said sliding cover slides along a portion of said tube during a needle retraction process.

12. The catheter assembly of claim 11 wherein said sliding cover slides along said tube portion to cover a tip portion of said needle when said needle is retracted into said tube.

13. The catheter assembly of claim 12 wherein said sliding cover locks into place when said needle is fully retracted into said tube.

14. The catheter assembly of claim 13 wherein said tube includes a catheter-gripping surface generally opposite said cover.

15. The catheter assembly of claim 14 wherein a portion of said tube proximate to said catheter-gripping surface provides a fluid shield during a needle retraction process.

16. A catheter system comprising:
a tube;
a needle carrier connected to a needle of a catheter assembly, said carrier being slidably received within said tube; and
a retraction spring connected at one end to said tube, said retraction spring being maintained in a stretched state while said needle carrier engages a tube projection, and said spring retracting said needle into said tube when said tube is rotated relative to said needle carrier to remove said tube projection engagement.

17. The catheter assembly of claim 16 further comprising a catheter-gripping area having a surface treatment to promote assembly manipulation.

18. The catheter assembly of claim 16 wherein said area includes a textured or raised surface or both and said area is located at a distal end of said tube.

19. The catheter assembly of claim 16 further comprising a sliding cover.

20. The catheter assembly of claim 19 wherein said sliding cover slides along a portion of said tube during a needle retraction process.

21. The catheter assembly of claim 19 wherein said sliding cover slides along said tube portion to cover a tip portion of said needle when said needle is retracted into said tube.

22. The catheter assembly of claim 21 wherein said sliding cover locks into place when said needle is fully retracted into said tube.

23. The catheter assembly of claim 22 wherein said tube includes a catheter-gripping surface generally opposite said cover.

24. The catheter assembly of claim 23 wherein a portion of said tube proximate to said catheter-gripping surface provides a fluid shield during a needle retraction process.
